(19) United States
(12) Patent Application Publication
(10) Pub. No.: US 2024/0359144 A1
(43) Pub. Date: Oct. 31, 2024

(54) AGGREGATE SEPARATION METHOD

(71) Applicant: TELIX PHARMACEUTICALS (INNOVATIONS) PTY LTD, North Melbourne, Victoria (AU)

(72) Inventors: Oliver William Sterritt, North Melbourne (AU); Michael Paul Wheatcroft, North Melbourne (AU)

(21) Appl. No.: 18/687,806

(22) PCT Filed: Sep. 1, 2022
(86) PCT No.: PCT/AU2022/051067

§ 371 Date: Feb. 28, 2024

(30) Foreign Application Priority Data
Sep. 1, 2021 (AU) 2021902839

(51) Int. Cl. B01D 71/16; B01D 69/02; C07K 1/34; C07K 16/30

(52) U.S. Cl. CPC B01D 71/16; B01D 69/02; C07K 1/34; C07K 16/3069; B01D 2315/10

(57) ABSTRACT

The disclosure provides methods of removing aggregate from a composition comprising a protein, an aggregate of the protein and a liquid carrier. The method involves subjecting the composition to one or more filtering steps comprising passing the composition through a cellulose acetate membrane to selectively adsorb at least some of the aggregate onto the membrane while substantially allowing the protein to pass through the membrane. Preferred protein compositions for aggregate removal comprise antibodies conjugated to chelating ligands.

### CROSS-REFERENCE TO RELATED APPLICATION

This application claims priority to Australian provisional application no. 2021902839 (filed on 1 Sep. 2021), the entire contents of which are incorporated herein by reference.

### FIELD OF THE INVENTION

This invention relates to a method of selectively removing a protein aggregate from a composition.

### BACKGROUND OF THE INVENTION

Protein aggregation is a common problem arising in handling proteins outside of their native environment. Aggregation can occur during handling steps or upon storage of a protein sample. Typically aggregates do not possess the same function as non-aggregated protein, and therefore aggregation represents one pathway to loss of function of a protein sample.

One class of protein where aggregation is a problem is protein conjugates. For example, protein conjugates with a chelating ligand are of continuing interest, for example, for their potential use as a therapeutic, diagnostic or theranostic agent.

Chelating ligands are typically multi-dentate and are preferably selective for a nuclide of therapeutic or diagnostic potential.

One problem arising in the preparation of protein conjugates particularly at commercial scale is the formation of aggregates due to the relatively harsh synthetic conditions required for various preparation steps. Protein aggregates may be solid aggregates or soluble aggregates.

Aggregate mitigation strategies include adapting the preparation procedures for the conjugates to reduce aggregate formation or purification procedures to separate aggregated protein from the protein conjugates.

There is therefore a continuing need to provide at least alternative processes for preparing proteins, including protein conjugates with chelating ligands, that can provide the desired proteins in meaningful yields with low levels of aggregate.

Reference to any prior art in the specification is not an acknowledgment or suggestion that this prior art forms part of the common general knowledge in any jurisdiction or that this prior art could reasonably be expected to be understood, regarded as relevant, and/or combined with other pieces of prior art by a skilled person in the art.

### SUMMARY OF THE INVENTION

In one aspect, the present invention provides a method of removing an aggregate of a protein from a composition comprising the protein and the aggregate of the protein in a liquid carrier. The method comprises subjecting the composition to one or more filtering steps comprising passing the composition through a cellulose acetate membrane to selectively adsorb the aggregate onto the membrane while substantially allowing the protein to pass through the membrane.

The present invention also provides a method of purifying a protein. The method comprises subjecting a composition comprising the protein and the aggregate of the protein in a liquid carrier to one or more filtering steps comprising passing the composition through a cellulose acetate membrane to selectively adsorb the aggregate onto the membrane while substantially allowing the protein to pass through the membrane.

The present invention also provides a protein obtainable or obtained by the methods described herein, and to compositions comprising the protein obtainable or obtained by the methods described herein.

### Selected Definitions

The term “alkyl” is intended to include saturated straight chain and branched chain hydrocarbon groups. In some embodiments, alkyl groups have from 1 to 12, 1 to 10, 1 to 8, 1 to 6, or from 1 to 4 carbon atoms. In some embodiments, alkyl groups have from 5-21, from 9-21, or from 11-21 carbon atoms, such as from 11, 13, 15, 17, or 19 carbon atoms. Examples of straight chain alkyl groups include, but are not limited to, methyl, ethyl, n-propyl, n-butyl, n-pentyl, n-hexyl, n-heptyl, and n-octyl. Examples of branched alkyl groups include, but are not limited to, isopropyl, iso-butyl, sec-butyl, tert-butyl, neopentyl, isopentyl, and 2,2-dimethylpropyl.

The term “halo” is intended to include chloro (—Cl), bromo (—Br), fluoro (—F) and iodo (—I) groups. In some embodiments, halo may be selected from chloro, bromo and fluoro, preferably fluoro.

As used herein, the term “theranostic” refers to the ability of compounds/materials to be used for diagnosis as well as for therapy. The term “theranostic reagent” relates to any reagent which is both suitable for detection, diagnostic and/or the treatment of a disease or condition of a patient. The aim of theranostic compounds/materials is to overcome undesirable differences in biodistribution and selectivity, which can exist between distinct diagnostic and therapeutic agents.

As used herein, the term “and/or” means “and”, or “or”, or both.

The term “(s)” following a noun contemplates the singular and plural form, or both.

As used herein, except where the context requires otherwise, the term “comprise” and variations of the term, such as “comprising”, “comprises” and “comprised”, are not intended to exclude further additives, components, integers or steps.

It is intended that reference to a range of numbers disclosed herein (for example, 1 to 10) also incorporates reference to all rational numbers within that range (for example, 1, 1.1, 2, 3, 3.9, 4, 5, 6, 6.5, 7, 8, 9, and 10) and also any range of rational numbers within that range (for example, 2 to 8, 1.5 to 5.5, and 3.1 to 4.7) and, therefore, all sub-ranges of all ranges expressly disclosed herein are hereby expressly disclosed. These are only examples of what is specifically intended and all possible combinations of numerical values between the lowest value and the highest value enumerated are to be considered to be expressly stated in this application in a similar manner.

Various features of the invention are described with reference to a certain value, or range of values. These values are intended to relate to the results of the various appropriate measurement techniques, and therefore should be interpreted as including a margin of error inherent in any particular measurement technique. Some of the values referred to herein are denoted by the term “about” to at least in part account for this variability. The term “about”, when used to describe a value, may mean an amount within +10%, +5%, +1% or +0.1% of that value.

Further aspects of the present invention and further embodiments of the aspects described in the preceding paragraphs will become apparent from the following description, given by way of example and with reference to the accompanying drawings.

### BRIEF DESCRIPTION OF THE DRAWINGS

FIG. 1. Graph illustrating estimated binding capacity of various cellulose acetate filter sizes under pre-tangential flow filtration (TFF) conditions.

FIG. 2. Graph illustrating estimated binding capacity of various cellulose acetate filter sizes under post-TFF conditions.

FIG. 3. Graph illustrating in-process size exclusion chromatography-high-performance liquid chromatography (SEC-HPLC) data for four good manufacturing practice (GMP) girentuximab-N-succinyl-desferrioxamine conjugate (GmAb-DFO) process batches including pre-TFF and post-TFF cellulose acetate filtration steps.

### DETAILED DESCRIPTION OF THE EMBODIMENTS

The invention relates to a method of removing an aggregate of a protein from a composition comprising the protein and the aggregate of the protein. The method comprises:
subjecting a composition comprising the protein and the aggregate of the protein in a liquid carrier to one or more filtering steps comprising passing the composition through a cellulose acetate membrane.

The present inventors have surprisingly found that cellulose acetate membranes (also referred to as cellulose acetate filters), while typically used for sterilising and removing particulate matter and bioburden from protein solutions, are also able to effectively remove aggregates from a liquid mixture containing protein and aggregates of the protein.

The membrane comprises cellulose acetate. Without being bound by theory, it is believed that the processes described herein may comprise interaction of the aggregates with the cellulose acetate of the membrane rather than filtration based on aggregate size. This interaction may contribute to the surprising result that cellulose acetate filters were able to selectively remove protein aggregates from a composition also comprising the monomeric protein where membranes of similar pore size but of different material were unable to remove the aggregates from the protein. The interaction of the aggregates and the cellulose acetate is believed to at least predominantly be adsorption of the protein aggregate onto the cellulose acetate. Accordingly, the processes may remove aggregate from the composition comprising the protein and the aggregate by the cellulose acetate membrane selectively adsorbing at least some of the aggregate onto the membrane while allowing the protein to pass through the membrane.

Also described herein is a method of removing an aggregate of a protein from a composition comprising the protein and the aggregate of the protein, the method comprising:
subjecting a composition comprising the protein and the aggregate of the protein in a liquid carrier to one or more filtering steps comprising passing the composition through a cellulose acetate membrane to thereby remove at least some of the aggregate from the composition.

It will be appreciated that the present invention relates to the ability of the cellulose acetate membrane to remove aggregate from a complex protein sample, as distinct from their conventional use for removing particulate matter and bioburden. Accordingly, in some embodiments, the methods and processes described herein are not for the removal of particulate matter and/or bioburden.

The cellulose acetate membrane may be any suitable size. In some embodiments, the cellulose acetate membrane has a size (also referred to as a filtration area) of about 0.015 m² to about 0.60 m², for example a size of about 0.015 m², about 0.03 m², about 0.05 m², about 0.10 m², about 0.15 m², about 0.20 m², about 0.25 m², about 0.30 m², about 0.35 m², about 0.40 m², about 0.45 m², about 0.50 m², about 0.55 m², or about 0.6 m². In some embodiments, the size may be any size from these values to any other value, for example a size of about 0.015 m² to about 0.1 m² or a size of from about 0.03 m² to about 0.10 m². In some embodiments, the cellulose acetate membrane has a size of 0.05 m². It will be appreciated that the cellulose acetate membrane described herein typically has a larger size than those conventionally used for removal of bioburden and particulate matter from protein samples.

The cellulose acetate membrane may be provided in any suitable form. By way of example, the cellulose acetate membrane may be in the form a centrifuge filter, a syringe filter, or a capsule filter, any of which may be suitable for process (gram) scale.

The cellulose acetate membrane may be suitable for use in a flow through (continuous) method or process. Accordingly, in some embodiments, each of the one or more filtering steps is independently conducted in flow through mode. As shown in the Examples, this may advantageously allow the cellulose acetate membrane described herein to be used in large scale protein manufacturing processes.

In some embodiments, the cellulose acetate membrane does not comprise or is substantially free of cellulose acetate nanoparticles. Bee et al (Journal of Pharmaceutical Sciences, Vol 98, No 9, 3218-3238) previously reported that protein aggregates exhibit an affinity to cellulose acetate nanoparticles. Advantageously, as shown in the Examples, although having a comparatively lower relative surface area than cellulose acetate nanoparticles, the cellulose acetate membrane described herein is capable of reducing aggregate content to an acceptable quality level.

The cellulose acetate membrane may comprise pores of any suitable size. In some embodiments, the cellulose acetate membrane comprises pores having an average diameter (also referred to as pore size) of about 0.2 μm to about 0.8 μm, for example about 0.2 μm, about 0.25 μm, about 0.3 μm, about 0.35, about 0.4 μm, about 0.45 μm, about 5.0 μm, about 5.5 μm, about 6.0 μm, about 6.5 μm, about 7.0 μm, about 7.5 μm, or about 8.0 μm. In some embodiments, the average diameter may be any average diameter from these values to any other value, for example 0.2 μm to about 0.45 μm. In some embodiments, the cellulose acetate membrane comprises pores having an average diameter of about 0.2 μm.

The liquid carrier (also referred to as the liquid mixture) in each filtering step may independently have a pH of about 4.0 to about 8.0, for example a pH of about 4.0, about 4.1, about 4.2, about 4.3, about 4.4, about 4.5, about 4.6, about 4.7, about 4.8, about 4.9, about 5.0, about 5.1. about 5.2, about 5.3, about 5.4, about 5.5, about 5.6, about 5.7, about 5.8, about 5.9, about 6.0, about 6.1, about 6.2, about 6.3, about 6.4, about 6.5, about 6.6, about 6.7, about 6.8, about 6.9, about 7.0, about 7.1, about 7.2, about 7.3, about 7.4 about 7.5, about 7.6, about 7.7, about 7.8, about 7.9, or about 8.0. In some embodiments, the pH may be any pH from these values to any other value, for example a pH of about 4.4 to about 7.4 or a pH of about 4.4 to about 5.9. Advantageously, in contrast to other techniques typically used for removing protein aggregates, such as chromatography resins, the use of the cellulose acetate membrane described herein may allow for removal of aggregate in a range of pH conditions.

The composition comprises the protein, aggregate of the protein and a liquid carrier. The composition may be a solution of the protein and aggregate in the liquid carrier, or the composition may be a suspension or emulsion of the protein and/or aggregate in the liquid carrier. In some embodiments, the protein is in solution with the liquid carrier and the aggregate is in suspension in the liquid carrier. Typically, the composition is a homogeneous mixture of the protein and aggregate in the liquid carrier. The composition may be in any form capable of being passed through the cellulose acetate membrane, and typically is a liquid composition.

The liquid carrier in each filtering step may independently be an aqueous solution, for example sodium chloride solution or a buffer solution. In some embodiments, the liquid carrier comprises a buffer solution. Any suitable buffer solution compatible with proteins may be used, for example phosphate buffered saline (PBS), 2-(N-morpholino) ethanesulfonic acid (MES-NaOH), disodium hydrogen phosphate, 3-(N-morpholino) propanesulfonic acid (MOPS-KOH), tris(hydroxymethyl)aminomethane hydrochloride (Tris-HCl) and N-(2-hydroxyethyl) piperazine-N′-(2-ethanesulfonic acid) (HEPES). In some embodiments, the buffer solution is PBS. Advantageously, in contrast to other techniques typically used for removing protein aggregates (e.g., chromatography resins which require specific buffer conditions), the use of the cellulose acetate membrane described herein may allow for removal of aggregate in a range of conditions.

In some embodiments, the one or more filtering steps comprises two or more filtering steps, for example two, three, four, five or more filtering steps. The number of filtering steps may be suitably selected depending on, for example, depending on the amount of aggregate in the starting composition and/or the size of the cellulose acetate membrane.

In some embodiments, the one or more filtering steps comprises two filtering steps. Accordingly, in some embodiments, the one or more filtering steps comprise:
a first filtering step comprising passing the composition through a cellulose acetate membrane; and
a second filtering step comprising passing the composition through a cellulose acetate membrane.

Described another way, in some embodiments, the method comprises:
providing a composition comprising a protein and an aggregate of the protein in a liquid;
subjecting the composition to a first filtering step comprising passing the composition through a cellulose acetate membrane to provide a first filtrate that is enriched in the protein relative to the aggregate compared to the composition prior to passing through the cellulose acetate membrane; and
subjecting the first filtrate to a second filtering step comprising passing the first filtrate through a cellulose acetate membrane to provide a second filtrate further enriched in the protein relative to the aggregate compared to the first filtrate.

The pH of the first filtrate may be different to the pH of the composition prior to the first filtering step. In some embodiments, the first filtrate has a pH of about 5.6 to about 5.9. In some embodiments, the method further comprises, prior to the second filtering step, adjusting the pH of the first filtrate.

It will be appreciated that the method described herein may apply to any protein (typically a monomer) which has the potential to form undesired aggregates known in the art. As used herein, the term “aggregate” will be understood to include high molecular weight (HMW) aggregates of the protein, typically multimers larger than a dimer. The aggregates may be insoluble aggregates that form particulates and may precipitate from the solution in which they are formed, or the aggregates may be soluble aggregates.

As used herein, the term “protein” will be understood to encompass protein conjugates, eg a protein to which another (non-protein) chemical moiety is linked typically by covalent bonding. Accordingly, in some embodiments, the protein is a protein conjugate. Described another way, in some embodiments, the protein comprises a conjugated chemical moiety, eg a (non-protein) chemical moiety linked to the protein. The chemical moiety (also referred to as a prosthetic group) may be any suitable chemical moiety known in the art. In some embodiments, the chemical moiety may be a chelating moiety.

In some embodiments, the protein and conjugated chemical moiety are linked directly through a covalent bond. In some embodiments, the protein and the conjugated chemical moiety are linked through a linking group.

In some embodiments, the linking group is a bifunctional linker. The bifunctional linker may be any diradical species capable of covalently linking the chemical moiety and the protein together. Suitable bifunctional linkers include bromoacetyl, thiols, succinimide ester (eg succinyl), tetrafluorophenyl (TFP) ester, a maleimide, amino acids (including natural and non-natural amino acids), a nicotinamide, a nicotinamide derivative, or using any amine or thiol-modifying chemistry known in the art. In some embodiments, the bifunctional linker is succinyl.

In some embodiments, the bifunctional linker comprises a chain of atoms defining a longest linear path of 2-10 atoms between the conjugated chemical moiety and the protein.

In some embodiments, the bifunctional linker may be a C₁₋₁₀alkyl or haloC₁₋₁₀alkyl optionally interrupted by one or more groups selected from: —O—, —NR—, —S—, —C(O)—, —C(O)O—, —C(O) NR—, —OC(O)—, —NRC(O)—, —OC(O)O—, —NRC(O)O—, —OC(O)NR—, —NRC(O) NR—, wherein R is selected from H and C₁₋₄alkyl.

In some embodiments, the protein (or the conjugated chemical moiety) comprises a chelating ligand, ie a chelating ligand linked to the protein. The chelating ligand may be any suitable chelator capable of chelating a metal ion known in the art.

In some embodiments, the chelating ligand is capable of chelating a radionuclide. Examples of suitable chelating ligands include TMT (6,6″-bis[N,N″,N′-tetra(carboxymethyl)aminomethyl)-4′-(3-amino-4-methoxyphenyl)-2,2′:6′,2″-terpyridine), DOTA (1,4,7,10-tetraazacyclododecane-N,N′,N″,N″-tetraacetic acid, also known as tetraxetan), TCMC (the tetra-primary amide of DOTA), DO3A (1,4,7,10-Tetraazacyclododecane-1,4,7-tris(acetic acid)-10-(2-thioethyl) acetamide), CB-DO2A (4,10-bis(carboxymethyl)-1,4,7,10-tetraazabicyclo[5.5.2]tetradecan), NOTA (1,4,7-triazacyclononane-triacetic acid)Diamsar (3,6, 10, 13, 16, 19-hexaazabicyclo[6.6.6]eicosane-1,8-diamine), DTPA (Pentetic acid or diethylenetriaminepentaacetic acid), CHX-A″-DTPA ([(R)-2-Amino-3-(4-isothiocyanatophenyl) propyl]-trans-(S,S)-cyclohexane-1,2-diamine-pentaacetic acid), EDTA (ethylenediamine tetraacetic acid), TETA (1,4,8,11-tetraazacyclotetradecane-1,4,8,11-tetraacetic acid), Te2A (4,11-bis(carboxymethyl)-1,4,8,11-tetraazabicyclo[6.6.2]hexadecane), HBED (N, N-bis(2-Hydroxybenzyl)ethylenediamine-N,N-diacetic acid), DFO (Desferrioxamine), and analogues or derivatives thereof such as DFO* and DFOsq (DFO-squaramide), HYNIC (6-hydrazinonicotinamide), and HOPO (3,4,3-(LI-1,2-HOPO), or other ligand as described herein, or a derivative thereof. Suitable derivatives include modification to non-coordinating portions of the molecule and may include functional group interconversion, such as the presence of an amide in place of a carboxyl group.

In some embodiments, the chelating ligand is DFO or an analogue thereof. DFO and its analogues (including DFO*, DFOsq, DFONCS, DFO*sq, and DFO*NCS) are selective chelating ligands for desired nuclides of therapeutic, diagnostic and/or theranostic potential. In particular, DFO and its analogues are selective chelators for ⁸⁹Zr. ⁸⁹Zr is a beta-positive emitter (av) (0.396 MeV) with a half-life extending to 3.3 days. ⁸⁹Zr has potential applications in positron emission tomography (PET) imaging and when included in a protein conjugate (such as those produced by the methods of the invention) is of particular interest in immunological PET (immuno-PET) imaging due to its extended 3.3 d half-life which matches the circulation half-life of an antibody. In immuno-PET imaging, tumours are imaged based upon expression of tumour-associated antigens on tumour cells through the use of a radionuclide complex conjugated to an appropriate antibody.

In some embodiments, the chelating ligand chelates a radionuclide. The radionuclide is preferably a radionuclide of therapeutic or diagnostic potential. Examples of suitable isotopes include: actinium-225 (²²⁵Ac), astatine-211 (²¹¹At), bismuth-212 and bismuth-213 (²¹²Bi, ²¹³Bi), copper-64 and copper-67 (⁶⁴Cu, ⁶⁷Cu), gallium-67 and gallium-68 (⁶⁷Ga and ⁶⁸Ga), indium-111 (¹¹¹In), iodine-123, -124, -125 or -131 (¹²³I, ¹²⁴I, ¹²⁵I, ¹³¹I) (¹²³I), lead-212 (²¹²Pb), lutetium-177 (¹⁷⁷Lu), radium-223 (²²³Ra), samarium-153 (¹⁵³Sm), scandium-44 and scandium-47 (⁴⁴Sc, ⁴⁷Sc), strontium-90 (⁹⁰Sr), technetium-99 (^{99m}Tc), yttrium-86 and yttrium-90 (⁸⁶Y, ⁹⁰Y), zirconium-89 (⁸⁹Zr).

One class of protein conjugates of particular interest are those where the protein moiety is able to localise the conjugate within a subject after administration to assist with imaging, eg by PET, SPECT or other suitable imaging technique. Accordingly, in some embodiments, the protein comprises or is a protein targeting agent.

As used herein, a “protein targeting agent” refers to any protein capable of:
1. stable conjugation with the conjugated chelating group (both in free form and when chelating a nuclide, such as a radionuclide),
2. forming deleterious aggregates during handling, and
3. localising within a subject following administration (eg the protein targeting agent may localise in one or more organs, organelles, cell-types or receptor-types).

The protein targeting agent may be a polypeptide, a protein (eg an antibody and its derivatives such as nanobodies, diabodies, antibody fragments) that is able to bind to a certain biological target or to express a certain metabolic activity.

Non-limiting examples of suitable targeting agents include molecules that target VEGF receptors, analogs of bombesin or GRP receptor targeting molecules, molecules targeting somatostatin receptors, RGD peptides or molecules targeting avp3 and avP5, annexin V or molecules targeting the apoptotic process, molecules targeting estrogen receptors, biomolecules targeting the plaque, molecules targeting prostate specific membrane antigen (PSMA), molecules targeting a carbonic anhydrase (such as carbonic anhydrase IX; CAIX).

In some embodiments, the protein comprises or is an antibody or a derivative thereof, including as nanobodies, diabodies, antibodies fragments and the like.

In any embodiment, the protein is an antibody or antigen binding fragment thereof, for binding to carbonic anhydrase IX (CAIX). An especially preferred antibody is cG250, preferably girentuximab (INN), also referred to herein as GmAb. Another especially preferred embodiment is the monoclonal antibody G250 produced by the hybridoma cell line DSM ACC 2526. The antibody cG250 is an lgG1 kappa light chain chimeric version of an originally murine monoclonal antibody mG250. The antibody of antigen binding fragment thereof may also be a humanised form of girentuximab. In particularly preferred embodiments, the antibody for binding to CAIX is one that is described in WO 2021/000017, the contents of which are hereby incorporated by reference.

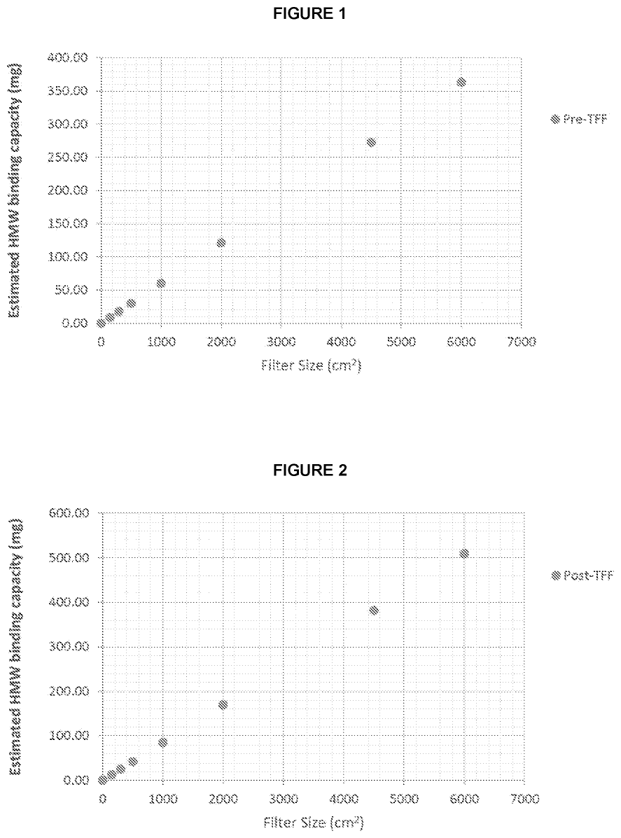

In any embodiment, the protein is an antibody, or antigen binding fragment thereof, for binding to prostate specific membrane antigen (PSMA), such as J591, or huJ591. Antibody J591 is described in Liu et al., Cancer Res 1997; 57:3629-34. The antibody or antigen-binding fragment thereof may have at least one, two and preferably three CDRs from: the heavy chain variable region of murine J591 (as defined in SEQ ID NO: 1, 2, and 3, and depicted in FIG. 1A of US20060088539, incorporated herein by reference); and the light chain variable region of murine J591 (see SEQ ID NO:4, 5 and 6, depicted in FIG. 1B of US20060088539, incorporated herein by reference). The antibody or antigen-binding fragment thereof can have the heavy variable and light chains of the J591 antibody, or any modified form thereof, as described in US20060088539, FIGS. 1A and 1B. The antibody or antigen-binding fragment thereof can have the heavy variable and light chains of a deimmunised J591 antibody, or any modified form thereof, as described in US20060088539, FIGS. 2A and 2B. In particularly preferred embodiments, the antibody for binding to PSMA is one that is described in WO 2021/000017, the contents of which are hereby incorporated by reference.

In some embodiments, the protein is an antibody or derivative thereof capable of targetting CAIX or PSMA. In some embodiments, the protein is selected from girentuximab and HuJ591, wherein the protein is optionally conjugated with a chelating ligand. In some embodiments, the protein comprises or is girentuximab (GmAb). GmAb is a monoclonal antibody to CAIX. In some embodiments, the protein comprises or is HuJ591. HuJ591 is a monoclonal antibody of PSMA.

In some embodiments, the protein comprises or is a polypeptide. The polypeptide may comprise a minimum sequence of at least about 20, 25 or 30 amino acid residues. The polypeptide may comprise up to about 35, 40, 45 or 50 amino acid residues. The polypeptide may comprise any amino acid sequence length from any of these minimum values to any maximum value, including for example about 20 to about 50 amino acid residues. Aggregation of peptide has been reviewed in Zapadka K L, Becher F J, Gomes dos Santos A L, Jackson S E. 2017 Factors affecting the physical stability (aggregation) of peptide therapeutics. Interface Focus 7:20170030. http://dx.doi.org/10.1098/rsfs.2017.0030, which is entirely incorporated herein by reference.

In some embodiments, the protein comprises or is a native protein and is isolated from its source. In some embodiments, the protein comprises synthetic or semi-synthetic residues, or the protein itself is synthetic or semi-synthetic. The protein (or protein moiety in the case of a protein conjugate) may be prepared by any means known in the art, including direct amino acid synthesis, recombinant technologies, and ligation of fragments to form the desired protein.

In some embodiments, the composition comprising protein and aggregate is obtained from a process for preparing a protein conjugate (eg a conjugate of a chelating ligand linked to a protein) in which an undesired aggregate is formed. One example of such a process is described for the preparation of DFO-GmAb conjugate in the Examples.

In some embodiments, the composition further comprises a dimeric protein, ie a dimer of the protein.

The composition comprising the protein before the one or more filtering steps (also referred to as the starting composition) may comprise at least about 10%, 11%, 12%, 13%, 14%, 15%, 16%, 17%, 18%, 19%, 20%, 21%, 22%, 23%, 24%, 25%, 26%, 27%, 28%, 29%, 30% or higher concentration of aggregate relative to the protein concentration, or relative to the total content of protein species in the composition (eg protein, aggregate, and dimer if present). The composition comprising the protein may comprise aggregate from any one of these percentages to any other percentage, for example from about 10% to about 25% or about 11% to about 19%. The concentration of aggregate relative to the protein (or total protein species) may be determined by SEC-HPLC and comparison of the area under the peak attributable to the aggregate species compared with the area under the peak for the monomeric protein (and other protein species if present).

The composition comprising the protein before the one or more filtering steps may comprise the protein in a concentration of at least about 74%, 75%, 76%, 77%, 78%, 79%, 80%, 81%, 82%, 83%, 84%, 85%, 86%, 87%, 88%, 89%, 90% or higher concentration of protein, relative to the aggregate, or relative to the total content of protein species in the composition (eg protein, aggregate, and dimer if present). The protein may be present in a concentration from any one of these percentages to any other percentage, for example from about 80% to about 90%. The concentration of protein may be determined in a similar manner to the concentration of aggregate species, for example by SEC-HPLC and comparison of the area under the peak for the respective relevant peaks.

The composition comprising the protein before the one or more filtering steps may further comprise dimeric protein. Typically the dimer is present in a concentration of not more than about 15%, 12.5%, 10%, 8%, 6%, 5%, 4%, 3%, 2%, 1% or lower concentration of dimer. The dimer may be present in a concentration from any one of these percentages to any other percentage, for example from about 2% to about 12.5% or about 3% to about 6%. The concentration of dimer may be determined in a similar manner to the concentration of aggregate species, for example by SEC-HPLC and comparison of the area under the peak for the respective relevant peaks.

The method may further comprise, prior to any one or more of the filtering steps, a step of subjecting the composition to a buffer exchange. Alternatively, in some embodiments, a step of subjecting a buffer exchange prior to any one of more of the filtering steps is not conducted. Advantageously, in contrast to other techniques typically used for removing protein aggregates, such as chromatography resins, the use of the cellulose acetate membrane described herein does not require a buffer exchange step.

In some embodiments, each of the filtering steps independently reduces the aggregate content in the composition to not more than about 25%, for example not more than about 20%, 15%, 12.5%, 10%, 9%, 8%, 7%, 6%, 5%, 4%, 3%, 2%, 1%, 0.9%, 0.8%, 0.7%, 0.6%, 0.5%, 0.4%, 0.3%, 0.2%, 0.1%, 0.05%, 0.01%, 0.005%, 0.001%, 0.0005%, 0.0001% or lower concentration of aggregate, relative to the protein or relative to the total content of protein species in the composition (eg protein, aggregate, and dimer if present). The aggregate content may be independently reduced from any one of these percentages to any other percentage, for example from about 0.001 to about 15% or about 0.1% to about 5%. In some embodiments, each of the filtering steps independently reduces the aggregate content in the composition to not more than about 5%. The concentration of aggregate relative to the protein (or total protein species) may be determined by SEC-HPLC and comparison of the area under the peak attributable to the aggregate species compared with the area under the peak for the monomeric protein (and other protein species if present).

In some embodiments, the methods may reduce aggregates of the protein by at least about 5 wt %, 10 wt %, 15 wt %, 20 wt %, 25 wt %, 30 wt %, 35 wt %, 40 wt % 45 wt %, 50 wt %, 60 wt %, 70 wt %, 80 wt %, 90 wt %, 95 wt %, or greater, based on the weight of aggregates present in the starting solution. The methods may reduce the aggregates from any of these percentages to any other of these percentages, for example the methods may reduce aggregates from the starting solution by about 5 wt % to about 95 wt % or about 10 wt % to about 40 wt % based on the weight of aggregates present in the starting solution.

In some embodiments, each of the filtering steps independently reduces the aggregate content in the composition by about 0.02 mg/cm² to about 0.15 mg/cm², for example about 0.02 mg/cm², about 0.03 mg/cm², about 0.04 mg/cm², about 0.05 mg/cm², about 0.06 mg/cm², about 0.07 mg/cm², about 0.08 mg/cm², about 0.09 mg/cm², about 0.10 mg/cm², about 0.11 mg/cm², about 0.12 mg/cm², about 0.13 mg/cm², about 0.14 mg/cm², or about 0.15 mg/cm², relative to the size of the cellulose acetate membrane. The aggregate content may be independently reduced from any one of these values to any other value, for example from about 0.03 mg/cm² to about 0.13 mg/cm² or from about 0.05 mg/cm² to about 0.10 mg/cm², relative to the size of the cellulose acetate membrane.

In some embodiments, each of the filtering steps independently reduces the aggregate content in the composition on average by about 0.05 mg/cm² to about 0.10 mg/cm², for example about 0.05 mg/cm², 0.06 mg/cm², 0.07 mg/cm², 0.08 mg/cm², 0.09 mg/cm², or 0.10 mg/cm², relative to the size of the cellulose acetate membrane. The aggregate content may be independently reduced on average from any one of these values to any other value, for example from about 0.06 mg/cm² to about 0.09 mg/cm², relative to the size of the cellulose acetate membrane.

In some embodiments, the cellulose acetate membrane has an average removal efficiency (also referred to as an average binding or filtering capacity) of about 0.05 mg aggregate at up to about 80% relative retention time (RRT)/cm² to about 0.10 mg aggregate at up to about 80% RRT/cm², for example about 0.05 mg, 0.06 mg, 0.07 mg, 0.08 mg, 0.09 mg, or 0.10 mg aggregate at up to about 80% RRT/cm², where RRT is relative to the SEC-HPLC peak attributable to the monomeric protein (i.e., the aggregate peak has an approximate retention time of up to about 80% of that of the monomeric protein peak). The average removal efficiency may be from any one of these values to any other value, for example from about 0.06 mg aggregate at up to about 80% RRT/cm² to about 0.09 mg aggregate at up to about 80% RRT/cm². The RRT may be any value up to about 80%, for example, about 80%, 70%, 60%, 50%, 40% or lower RRT. The RRT may be from any one of these values to any other value, for example from about 40% to about 80%, or from about 60% to about 80%. In some embodiments, the RRT is about 70% RRT. It will be appreciated that the dimeric protein peak typically has a RRT of about 85%.

Another aspect provides a method of purifying a protein, the method comprising:
subjecting a composition comprising a protein and an aggregate of the protein in a liquid carrier to one or more filtering steps comprising passing the composition through a cellulose acetate membrane to selectively adsorb at least some of the aggregate onto the membrane while allowing the protein to pass through the membrane.

Also provided herein is a method of purifying a protein, the method comprising:
subjecting a composition comprising a protein and an aggregate of the protein in a liquid carrier to one or more filtering steps comprising passing the composition through a cellulose acetate membrane to thereby remove at least some of the aggregate.

As used herein, the term “purifying” will be understood to mean that the aggregate content in the composition is reduced relative to the aggregate content prior to conducting the method.

Another aspect relates to the protein (also referred to as a purified protein) obtainable or obtained by the methods described herein.

Another aspect provides a composition comprising the protein (or purified protein) obtainable or obtained by the methods described herein.

The composition comprising the protein obtained or obtainable by the methods described herein (also referred to as the final or purified composition) may comprise not more than about 20%, 15%, 12.5%, 10%, 9%, 8%, 7%, 6%, 5%, 4%, 3%, 2%, 1% 1%, 0.9%, 0.8%, 0.7%, 0.6%, 0.5%, 0.4%, 0.3%, 0.2%, 0.1%, 0.05%, 0.01%, 0.005%, 0.001%, 0.0005%, 0.0001% or lower concentration of aggregate relative to the protein concentration, or relative to the total content of protein species in the composition (eg protein, aggregate, and dimer if present). The composition comprising the protein may comprise aggregates from any of these percentages to any other percentage, for example from about 0.01% to about 5%. In some embodiments, the composition comprises not more than about 5% aggregate content. The concentration of aggregate relative to the protein (or total protein species) may be determined by SEC-HPLC and comparison of the area under the peak attributable to the aggregate species compared with the area under the peak for the monomeric protein (and other protein species if present).

The composition obtained or obtainable by the methods described herein may comprise the protein in a concentration of at least 90%, for example at least 91%, 92%, 93%, 94%, 95% 96%, 97%, 98%, 99%, or 100%, relative to the aggregate concentration, or relative to the total content of protein species in the composition (eg protein, aggregate, and dimer if present). The protein may be present between any of these concentrations, for example from about 90% to about 100% or from about 95% to about 100%. The concentration of protein may be determined in a similar manner to the concentration of aggregate species, for example by SEC-HPLC and comparison of the area under the peak for the respective relevant peaks.

In some embodiments, the composition obtained or obtainable by the methods described herein may further comprise dimeric protein. Typically the dimer is present in a concentration of not more than about 1.5%, 1.4%, 1.3%, 1.2% 1.1%, 1%, 0.9%, 0.8% or 0.7%. The dimer may be present between any of these concentrations, for example from about 0.7% to about 1%. The concentration of dimer may be determined in a similar manner to the concentration of aggregate species, for example by SEC-HPLC and comparison of the area under the peak for the respective relevant peaks.

The composition comprising the protein typically comprises a liquid carrier. The liquid carrier may be any liquid carrier described herein. In some embodiments, the liquid carrier is an aqueous solution, for example sodium chloride solution or a buffer solution. In preferred embodiments, the liquid carrier comprises a buffer solution, such as those described herein.

Another aspect relates to a process for preparing a conjugate of a chelating ligand linked with a protein, the process comprising:
removing a metal from a metal complexed conjugate comprising a chelating ligand complexed to the metal linked with the protein, under conditions that induce the formation of an aggregate of the protein, to thereby provide a composition of the conjugate of the chelating ligand linked with the protein that is substantially free of chelated metal ion and the aggregate; and
subjecting the composition to one or more filtering steps comprising passing the composition through a cellulose acetate membrane to selectively adsorb at least some of the aggregate onto the membrane while allowing the conjugate of the chelating ligand linked with the protein to pass through the membrane and/or thereby remove at least some of the aggregate from the composition.

The protein and chelating ligand may be any of those described herein.

In one embodiment, the process is for preparing a conjugate of a desferrioxamine chelating ligand linked with a protein, the process comprising:
removing iron from an iron complexed conjugate comprising a desferrioxamine chelating ligand complexed to iron linked with the protein, under conditions that induce the formation of an aggregate of the protein, to thereby provide a composition comprising the conjugate of the desferrioxamine chelating ligand linked with the protein that is substantially free of chelated iron and the aggregate; and
subjecting the composition to one or more filtering steps comprising passing the composition through a cellulose acetate membrane to selectively adsorb at least some of the aggregate onto the membrane while allowing the conjugate of the desferrioxamine chelating ligand linked with the protein to pass through the membrane and/or thereby removing at least some of the aggregate from the composition.

The one or more filtering steps independently provide a composition in which the aggregate content is reduced, relative to the aggregate content before the respective filtering step, as described herein. In some embodiments, the process comprises two or more of these filtering steps, for example two, three, four, five or more filtering steps.

The process may further comprise a step of forming the metal complexed conjugate comprising the chelating ligand complexed to the metal linked with the protein. The forming step may comprise coupling a chelating ligand complexed to a metal ion with a protein. This step may be carried out by any known conjugation techniques known in the art. The metal chelated chelating ligand may be linked with the protein directly, or these moieties may be linked through a linking group, as described herein.

The process may further comprise a step of subjecting the composition to ultrafiltration and diafiltration (UFDF), for example by using a TFF system. In these embodiments, each of the one or more filtering steps may be independently conducted before UFDF, after UFDF, or both (in this case, the process comprises two, or two or more, of the filtering steps). Accordingly, in some embodiments, at least one of the one or more filtering steps is conducted before subjecting the composition to UFDF. Alternatively, or additionally, in some embodiments, at least one of the one or more filtering steps is conducted after subjecting the composition to UFDF. In preferred embodiments, the process comprises two (or two or more) filtering steps, and at least one of the filtering steps is conducted before subjecting the composition to UFDF, and at least one other of the one or more filtering steps is conducted after subjecting the composition to UFDF.

Accordingly, in some embodiments, the process comprises:
removing iron from an iron complexed conjugate comprising a desferrioxamine chelating ligand complexed to iron linked with the protein, under conditions that induce the formation of an aggregate of the protein, to thereby provide a composition comprising the conjugate of the desferrioxamine chelating ligand linked with the protein that is substantially free of chelated iron and the aggregate;
subjecting the composition to a first filtering step comprising passing the composition through a cellulose acetate membrane to selectively adsorb at least some of the aggregate onto the membrane while allowing the conjugate of the desferrioxamine chelating ligand linked with the protein to pass through the membrane and/or thereby remove at least some of the aggregate from the composition;
subjecting the composition to ultrafiltration and diafiltration; and
subjecting the composition to a second filtering step comprising passing the composition through a cellulose acetate membrane to selectively adsorb at least some of the aggregate onto the membrane while allowing the conjugate of the desferrioxamine chelating ligand linked with the protein to pass through the membrane and/or thereby remove at least some of the aggregate from the composition.

In these processes, the composition subjected to UFDF is the filtrate of the first filtering step, and the composition subjected to the second filtering step is the filtrate of the UFDF.

The conjugate of desferrioxamine chelating ligand linked with protein may be prepared as a bulk drug substance (BDS). Typically, the final steps in preparing a BDS involve sterilising-grade filtration, formulation and filling. In preferred embodiments, the one or more filtering steps are conducted prior to the final sterilising-grade filtration step. Described another way, in preferred embodiments, the one or more filtering steps are intermediate purification steps in the process.

Another aspect relates to the conjugate of chelating ligand linked with protein (also referred to as a purified conjugate of chelating ligand linked with protein) obtainable or obtained by the process described herein.

Another aspect provides a formulation comprising the conjugate of chelating ligand linked with protein (or purified conjugate of chelating ligand linked with protein) obtainable or obtained by the process described herein.

Also provided herein is a cellulose acetate membrane for removing an aggregate of a protein from a composition comprising the protein, the aggregate of the protein and a liquid carrier.

Further provided herein is a cellulose acetate membrane for use in removing an aggregate of a protein from a composition comprising the protein, the aggregate of the protein and a liquid carrier.

Further provided herein is a cellulose acetate membrane when used for removing an aggregate of a protein from a composition comprising the protein, the aggregate of the protein and a liquid carrier.

Further provided herein is use of a cellulose acetate membrane for removing an aggregate of a protein from a composition comprising the protein, the aggregate of the protein and a liquid carrier.

Also provided herein is a cellulose acetate membrane for selectively adsorbing an aggregate of a protein from a composition comprising the protein, the aggregate of the protein and a liquid carrier.

Further provided herein is a cellulose acetate membrane for use in selectively adsorbing an aggregate of a protein from a composition comprising the protein, the aggregate of the protein and a liquid carrier.

Further provided herein is a cellulose acetate membrane when used for selectively adsorbing an aggregate of a protein from a composition comprising the protein, the aggregate of the protein and a liquid carrier.

Further provided herein is use of a cellulose acetate membrane for selectively adsorbing an aggregate of a protein from a composition comprising the protein, the aggregate of the protein and a liquid carrier.

These cellulose acetate membranes may have any one or more features of the cellulose acetate membrane described herein.

These cellulose acetate membranes may be for, for use in, and/or when used for any aspect or embodiment of a process described herein.

In some embodiments, the cellulose acetate membrane described herein is used in a flow through method or process. In some embodiments, the cellulose acetate membrane is not used for removal of particulate matter and/or bioburden from the composition.

The present invention may provide one or more of the following advantages:

The methods advantageously reduce aggregate content to an acceptable quality level.

The methods advantageously allow for high (>85%-95%) protein monomer recovery.

The methods advantageously are scalable.

The methods may be applied to large scale (gram scale) industrial processes.

The methods may be automated.

It will be understood that the invention disclosed and defined in this specification extends to all alternative combinations of two or more of the individual features mentioned or evident from the text or drawings. All of these different combinations constitute various alternative aspects of the invention.

### EXAMPLES

The invention will be further described by way of non-limiting examples. It will be understood to persons skilled in the art of the invention that many modifications may be made without departing from the spirit and scope of the invention.

### Example 1. Filter Evaluation—Small Scale

To evaluate whether filter membrane materials could filter HMW aggregates from samples, two different filter types were tested and compared to results obtained when the sample was not filtered prior to analysis. GmAb-DFO samples from various different lots were diluted 90%:10% sample:diluent. A minimum of 250 μL of each sample type was passed through each filter membrane.

The following filters were evaluated:
Filter with cellulose acetate membrane, pore size 0.2 μm (Millipore Sigma; product no CLS8161-100EA)
Filter with polyvinylidene difluoride (PVDF) membrane, pore size 0.2 μm

The samples were evaluated by SEC-HPLC using an Agilent 1200 series HPLC system equipped with a Yarra SEC-300 (3 μm, 290 Å, 7.8×300 mm) SEC-HPLC column. Data were analysed by comparing the area of each protein species to the total area detected.

The results are provided in Table 1. The results show that filtering the sample with a cellulose acetate membrane provided a lower percentage of high molecular weight aggregate species (% HMW) in the filtrate and a higher percentage of monomeric protein, compared to both unfiltered samples and samples filtered with a PVDF filter. This result suggests that the unique chemical properties of the cellulose acetate membrane result in selective removal of aggregate over monomeric protein. This provides an indication that a cellulose acetate filter may be useful for removing protein aggregates from complex protein samples.

TABLE 1
Filter evaluation

| | % HMW | | | |
|---|---|---|---|---|
| Filter | RRT ~70% | Dimer (%) | Monomer (%) | LMW (%) |
| Cellulose acetate | 6.42 | 1.83 | 91.24 | 0.51 |
| filter | 5.52 | 1.03 | 92.67 | 0.75 |
| PVDF filter | 7.61 | 1.70 | 90.08 | 0.51 |
| | 6.95 | 1.00 | 91.28 | 0.73 |
| No filter | 8.06 | 1.80 | 89.64 | 0.49 |
| | 7.55 | 0.99 | 90.72 | 0.70 |

For comparison, other techniques typically used for aggregate removal were also evaluated, namely chromatography resins Eshmuno HCX, Toyopearl hexyl, Toyopearl NH2-750F, POROS 50 HS and Capto Adhere, Sartobind phenyl membrane, and ammonium sulfate fractionation. Table 2 sets out the techniques and their respective buffers. The samples were exchanged into the buffers indicated. Before loading onto the respective resins, the samples were analysed by absorbance at 280 nm (A280) and SEC-HPLC. The samples were then loaded onto each pre-equilibrated resin or membrane and incubated at ambient temperature. The resins and membranes were washed and the flow through collected. The flow through was then analysed by A280 and SEC-HPLC to determine % monomeric recovery and % HMW aggregate removal.

TABLE 2
Evaluation of other techniques for aggregate removal

| | | % | % HMW | Significant |
|---|---|---|---|---|
| | | Monomer | aggregate | dimer |
| Technique | Buffer | recovery | removal | increase? |
| Eshmuno | 0.15M sodium chloride, | 80% | 44% | No |
| HCX Resin | 25 mM sodium | | | |
| | phosphate, pH 8.0 | | | |
| Toyopearl | 0.7M Sodium Chloride, | 47% | 21% | No |
| Hexyl Resin | 25 mM Sodium | | | |
| | Phosphate, pH 6.0 | | | |
| Toyopearl | 0.9% Saline solution | 87% | 35% | Yes |
| NH2-750F | | | | |
| POROS 50 | 0.9% Saline solution | 87% | 49% | Yes |
| HS Resin | | | | |
| Capto Adhere | 0.15M Sodium Chloride, | 100% | 18% | No |
| Resin | 25 mM Sodium | | | |
| | Phosphate, pH 6.0 | | | |
| Sartobind | 0.5M Sodium Chloride, | 94% | 95% | Yes |
| Phenyl | 25 mM Sodium | | | |
| Membrane | Phosphate, pH 6.0 | | | |
| Ammonium | 1M Ammonium Sulfate, | 95% | 99% | No |
| Sulfate | 25 mm Sodium | | | |
| Fractionation | Phosphate, pH 7.0 | | | |

The results are provided in Table 2. Generally, the various techniques exhibited high monomeric recovery with low aggregate removal. Some techniques also exhibited significant increase in the dimeric protein. While the ammonium sulfate fractionation exhibited good results, this technique requires the addition of 1M ammonium sulfate, which may be deemed unacceptable and a process risk. The Sartobind phenyl membrane exhibited good monomeric recovery and high aggregate removal, but there was a significant increase in dimer. Another drawback to this and other chromatography resins is the need for an additional buffer exchange step to put the sample into the “chromatography” buffer. The chromatography resins require these precise conditions to be able to bind and remove the HMW aggregate from the protein composition, i.e., they have a narrow window of operating buffer conditions necessitating the buffer exchange step. The additional step would add to process time and also material loss (lower yield).

### Example 2. Cellulose Acetate Filter Evaluation—Large Scale

Cellulose acetate filters (Cellulose Acetate 0.2 μm filter Sartobran, Sartorius, Cat: 11107-25-N) were evaluated for removing aggregates produced during a process for preparing a conjugate of an antibody and desferrioxamine (DFO), namely girentuximab-N-succinyldesferrioxamine (GmAb-DFO). One example of a typical process for preparing GmAb-DFO involves the following steps: N-succinyldesferal: Fe(III) tetrafluorophenyl ester, (TFP-N-sucDf-Fe, syn: DFOTFP) was conjugated to chimeric girentuximab (GmAb) through the amidation of random lysine residues exposed on the antibody surface. Chelated iron was removed by transchelation with an excess amount of ethylenediaminetetraacetic acid (EDTA) at mild temperature (35° C.) and pH 4.4. The unreacted linker and other small molecules were removed by TFF resulting in conjugated GmAb-DFO. These procedural steps induced the formation of high molecular weight aggregate species.

### Binding Capacity Study

### Sample Preparation

Table 3 outlines the samples prepared for the study. Samples were clear and free from particulate matter before being loaded onto the 0.2 μm cellulose acetate filter. GmAb-DFO material from four different manufacturing lots were evaluated: lot #1; lot #2; lot #3; lot #4. Materials from each lot were thawed, pooled separately by batch for a total of four initial samples, and 0.2 μm filtered by a polyethersulfone (PES) membrane (Acrodisc) prior to each experimental execution. A 1:1 mixture of lot #3 and lot #4 was prepared and homogenised to prepare the middle point sample. The selected levels for the binding capacity study are displayed in Table 3.

TABLE 3
Sample preparation

| | | Sample | Sample | Loading | Volumetric |
|---|---|---|---|---|---|
| | | initial | volume | capacity | filter |
| | | conc | (mL | (g | throughput |
| Run | Material | (mg/ | GmAb- | GmAb- | (L GmAb- |
| no | Lot no | mL) | DFO) | DFO/m²) | DFO/m²) |
| 1 | Lot #1 | 2.1 | 2.4 | 11.2 | 5.34 |
| 2 | Post-TFF | | 4.8 | 22.4 | 10.68 |
| 3 | | | 6.0 | 28.0 | 13.35 |
| 4 | Lot #4 | 2.1 | 2.4 | 11.2 | 5.34 |
| 5 | Post-TFF | | 4.8 | 22.4 | 10.68 |
| 6 | | | 6.0 | 28.0 | 13.35 |
| 7 | Lot #3/Lot #4 | 2.1 | 4.8 | 22.4 | 10.68 |
| | 1:1 mixture | | | | |
| 8 | Pre-TFF | 1.5 | 3.36 | 11.2 | 7.47 |
| 9 | material | | 7.06 | 22.4 | 15.7 |
| 10 | Run 1 (Lot #1) | | 8.40 | 28.0 | 18.7 |
| 11 | Pre-TFF | 1.5 | 3.36 | 11.2 | 7.47 |
| 12 | material | | 7.06 | 22.4 | 15.7 |
| 13 | Run 2 (Lot #2) | | 8.40 | 28.0 | 18.7 |

### Pre-TFF Samples

In order to generate the pre-TFF samples, the GmAb-DFO materials (Lot #1 and Lot #2) were buffer exchanged into the pre-TFF sample buffer. The pre-TFF sample buffer was prepared by performing a blank conjugation run using PBS pH 7.1 in place of the GmAb starting material. The heating step was not included in the pre-TFF sample buffer generation since the step has no impact on the buffer composition. The PBS was adjusted to pH 9.6, then the solution was combined with linker at the molar ratio of 3:1 and kept for 30 min under gentle mixing at room temperature. The pH of the vessel was then brought to pH 4.4 using acid. As a final step, EDTA disodium was added, and the buffer was incubated for the final pH was adjusted to 7.0.

The sample buffer exchange was performed using PD-10 desalting columns. The buffer exchange was made using the following standard procedures. For the elution step, a vessel was placed under the columns to collect each sample. The elution of each sample was performed with 3.5 mL of pre-TFF sample buffer and collected.

### Binding Capacity Experimental Procedure

The samples of Table 3 were filtered using a 0.2 μm cellulose acetate filter. Characterisation of the flow through material was performed by SEC-HPLC and A280. Two aliquots were taken from each flowthrough material. The first aliquot was stored at 2-8° C. for characterisation while the second one was placed in the −80° C. freezer for 24 hours and then transferred to a −20° C. freezer until further characterisation.

The following protocol was executed during the binding capacity experiment. The protein solution pool was prepared according to the procedure described in the sample preparation section. The corresponding volume of Lot #1, Lot #4 and Lot #3/Lot #4 mixture was passed through a Sartorious 0.2 μm filter (Cat: 11107-25 -N) collecting the filtered protein solution for further characterisation. The cellulose acetate filter was pre-wet with PBS pH 7.1, the sample was passed through, and then the filter was flushed with PBS pH 7.1 (1.5× system-filter hold-up volume, the volume retained in the system and filter without air purge) to ensure complete recovery of the unbound species.

### Binding Capacity Study Results

### Pre-TFF Filtration Step

### % HMW and Dimer Reduction

The SEC-HPLC and A280 characterisation of the flowthrough material for the pre-TFF samples is displayed in Table 4. The percent reduction for HMW at 70% RRT and dimer were normalised as a percentage reduction based on the SEC-HPLC HMW aggregate and dimer data. In Table 4, the loading capacity was converted from mg of GmAb-DFO/m² to mg of HMW at 70% RRT/cm² since this is the major species that interacts with the cellulose acetate membrane, where 70% RRT means that the HMW aggregate peak has an approximate retention time of 70% of that of the monomeric peak.

TABLE 4
HMW and dimer reduction results for pre-TFF samples

| | Loading | | | | | Reduction | |
|---|---|---|---|---|---|---|---|
| | capacity | | | | | % | |
| | (mg HMW | % HMW | | | | HMW | |
| | at 70% | at 70% | | Dimer | Monomer | at 70% | % |
| Sample ID | RRT/cm²) | RRT (%) | LMW (%) | (%) | (%) | RRTª | dimer |
| Pre-TFF | 0.185 | 17.46 | 0.33 | 2.53 | 79.46 | 12.9% | −2.4% |
| Material Run | | | | | | | |
| 1 (Lot #1) | | | | | | | |
| Control | | | | | | | |
| Sample 1 | | | | | | | |
| Pre-TFF | | 15.20 | 0.35 | 2.59 | 81.65 | | |
| Material Run | | | | | | | |
| 1 (Lot #1) | | | | | | | |
| 0.2 μM CA | | | | | | | |
| Filtered | | | | | | | |
| Sample 1 | | | | | | | |
| Pre-TFF | 0.396 | 17.78 | 0.35 | 2.55 | 79.11 | 10.8% | −8.2% |
| Material Run | | | | | | | |
| 1 (Lot #1) | | | | | | | |
| Control | | | | | | | |
| Sample 2 | | | | | | | |
| Pre-TFF | | 15.86 | 0.35 | 2.76 | 80.80 | | |
| Material Run | | | | | | | |
| 1 (Lot #1) | | | | | | | |
| 0.2 μM CA | | | | | | | |
| Filtered | | | | | | | |
| Sample 2 | | | | | | | |
| Pre-TFF | 0.488 | 18.41 | 0.35 | 2.54 | 78.49 | 17.6% | −3.1% |
| Material Run | | | | | | | |
| 1 (Lot #1) | | | | | | | |
| Control | | | | | | | |
| Sample 3 | | | | | | | |
| Pre-TFF | | 15.17 | 0.37 | 2.62 | 81.62 | | |
| Material Run | | | | | | | |
| 1 (Lot #1) | | | | | | | |
| 0.2 μM CA | | | | | | | |
| Filtered | | | | | | | |
| Sample 3 | | | | | | | |
| Pre-TFF | 0.078 | 7.81 | 0.29 | 1.99 | 89.74 | 30.6% | −3.5% |
| Material Run | | | | | | | |
| 2 (Lot #2) | | | | | | | |
| Control | | | | | | | |
| Sample 1 | | | | | | | |
| Pre-TFF | | 5.42 | 0.3 | 2.03 | 92.06 | | |
| Material Run | | | | | | | |
| 2 (Lot #2) | | | | | | | |
| 0.2 μM CA | | | | | | | |
| Filtered | | | | | | | |
| Sample 1 | | | | | | | |
| Pre-TFF | 0.177 | 8.38 | 0.28 | 1.99 | 89.18 | 12.8 | −2.0% |
| Material Run | | | | | | | |
| 2 (Lot #2) | | | | | | | |
| Control | | | | | | | |
| Sample 2 | | | | | | | |
| Pre-TFF | | 7.31 | 0.28 | 2.03 | 90.21 | | |
| Material Run | | | | | | | |
| 2 (Lot #2) | | | | | | | |
| 0.2 μM CA | | | | | | | |
| Filtered | | | | | | | |
| Sample 2 | | | | | | | |
| Pre-TFF | 0.214 | 8.52 | 0.28 | 1.98 | 89.06 | 19.2% | −3.0% |
| Material Run | | | | | | | |
| 2 (Lot #2) | | | | | | | |
| Control | | | | | | | |
| Sample 3 | | | | | | | |
| Pre-TFF | | 6.88 | 0.28 | 2.04 | 90.63 | | |
| Material Run | | | | | | | |
| 2 (Lot #2) | | | | | | | |
| 0.2 μM CA | | | | | | | |
| Filtered | | | | | | | |
| Sample 3 | | | | | | | |

${}^{a} \% H M W at 70 \% RRT reduction = 100 - ( \frac{\% H M W at 70 \% RRT post ‐ filtration}{\% H M W at 70 \% RRT pre ‐ filtration} \times 100 )$

As shown in Table 4, a difference in the reduction of the HMW at 70% RRT between samples from different batches was observed, mainly due to the differences in the feed composition (i.e., sample composition, sample concentration, HMW aggregate content). The reduction in HMW at 70% RRT for Lot #1 samples ranged from 10.8% to 17.6% for loading conditions between 0.185 and 0.488 mg of HMW at 70% RRT/cm². For the Lot #2 material, a higher reduction in HMW at 70% RRT was observed, ranging from 12.8% to 30.6% for a loading capacity between 0.078 and 0.214 mg of HMW at 70% RRT/cm².

Despite the differences observed, a comparable reduction in % HMW at 70% RRT was observed for both the Lot #1 and Lot #2 samples when a similar quantity of HMW at 70% RRT/cm² was loaded onto the membrane (i.e., a 13% reduction of HMW at 70% RRT was observed for 0.185 mg HMW at 70% RRT (Lot #1)/cm² and 0.177 mg HMW at 70% RRT (Lot #2)/cm²).

The highest percent reduction of HMW at 70% RRT was observed for the experiment performed with a loading capacity of 0.078 mg HMW at 70% RRT/cm² for the Lot #2 sample, resulting in a reduction of 31% HMW at 70% RRT.

No significant differences in dimer content were observed across all the conditions studied. The differences in dimer observed between the pre- and post-filtration samples may be due to the slight enrichment of this species due to the removal of the HMW aggregate by the cellulose acetate filter.

### Overall Recovery and Monomer Recovery

Table 5 outlines the overall recovery and the monomer recovery obtained for each pre-TFF condition evaluated. The monomer percent recovery was calculated based on the mg of the monomer obtained pre-filtration and post-filtration using the purity result from the SEC-HPLC monomer read.

TABLE 5
Recovery results for pre-TFF samples

| | Loading | | | | | | |
|---|---|---|---|---|---|---|---|
| | Capacity | | Protein | | | | |
| | (g GmAb- | Volume | conc | Qty | | Recovery (%) | |
| Sample ID | DFO/m²) | (mL) | (mg/mL) | (mg) | Monomer | Overall | Monomer |
| Pre-TFF | 11.2 | 3.36 | 1.421 | 4.77 | 79.46 | 87.73% | 90.15% |
| Material Run 1 | | | | | | | |
| (Lot #1) | | | | | | | |
| Pool Sample 1 | | | | | | | |
| (Load) | | | | | | | |
| Pre-TFF | | 5.44 | 0.770 | 4.19 | 81.65 | | |
| Material Run 1 | | | | | | | |
| (Lot #1) | | | | | | | |
| 0.2 μm CA | | | | | | | |
| Filtered | | | | | | | |
| Sample 1 | | | | | | | |
| Pre-TFF | 22.4 | 7.06 | 1.421 | 10.03 | 79.11 | 92.99% | 94.98% |
| Material Run 1 | | | | | | | |
| (Lot #1) | | | | | | | |
| Pool Sample 2 | | | | | | | |
| (Load) | | | | | | | |
| Pre-TFF | | 9.32 | 1.001 | 9.33 | 80.80 | | |
| Material Run 1 | | | | | | | |
| (Lot #1) | | | | | | | |
| 0.2 μm CA | | | | | | | |
| Filtered | | | | | | | |
| Sample 2 | | | | | | | |
| Pre-TFF | 28 | 8.40 | 1.421 | 11.94 | 78.49 | 88.00% | 91.51% |
| Material Run 1 | | | | | | | |
| (Lot #1) | | | | | | | |
| Pool Sample 3 | | | | | | | |
| (Load) | | | | | | | |
| Pre-TFF | | 9.91 | 1.060 | 10.50 | 81.62 | | |
| Material Run 1 | | | | | | | |
| (Lot #1) | | | | | | | |
| 0.2 μm CA | | | | | | | |
| Filtered | | | | | | | |
| Sample 3 | | | | | | | |
| Pre-TFF | 11.2 | 3.36 | 1.343 | 4.51 | 89.74 | 90.38% | 92.72% |
| Material Run 2 | | | | | | | |
| (Lot #2) | | | | | | | |
| Pool Sample 1 | | | | | | | |
| (Load) | | | | | | | |
| Pre-TFF | | 5.61 | 0.727 | 4.08 | 92.06 | | |
| Material Run 2 | | | | | | | |
| (Lot #2) | | | | | | | |
| 0.2 μm CA | | | | | | | |
| Filtered | | | | | | | |
| Sample 1 | | | | | | | |
| Pre-TFF | 22.4 | 7.06 | 1.343 | 9.48 | 89.18 | 94.45% | 95.54% |
| Material Run 2 | | | | | | | |
| (Lot #2) | | | | | | | |
| Pool Sample 2 | | | | | | | |
| (Load) | | | | | | | |
| Pre-TFF | | 9.11 | 0.983 | 8.96 | 90.21 | | |
| Material Run 2 | | | | | | | |
| (Lot #2) | | | | | | | |
| 0.2 μm CA | | | | | | | |
| Filtered | | | | | | | |
| Sample 2 | | | | | | | |
| Pre-TFF | 28 | 8.40 | 1.343 | 11.28 | 89.06 | 91.02% | 92.62% |
| Material Run 2 | | | | | | | |
| (Lot #2) | | | | | | | |
| Pool Sample 3 | | | | | | | |
| (Load) | | | | | | | |
| Pre-TFF | | 10.33 | 0.994 | 10.27 | 90.63 | | |
| Material Run 2 | | | | | | | |
| (Lot #2) | | | | | | | |
| 0.2 μm CA | | | | | | | |
| Filtered | | | | | | | |
| Sample 3 | | | | | | | |

No significant difference in terms of monomeric recovery was observed between each loading condition evaluated for the cellulose acetate filtration. The percent recoveries ranged from 87.73% to 94.45%, with monomeric % recoveries ranging from 90.15% to 95.54%. High monomeric recoveries were observed for all of load conditions evaluated, indicating that monomeric GmAb-DFO did not bind to the cellulose acetate membrane at any significant level. This may provide an indication that that the losses associated with cellulose acetate membrane filtration may be related to the system hold up volume of the bench scale filters (the volume retained in the system and filter without air purge).

### Binding Capacity Results

The total binding capacity for HMW at 70% RRT of the cellulose acetate membrane used in this study was measured in batch mode and was referred to as the maximum amount of HMW at 70% RRT bound to the membrane under the feed and buffer conditions evaluated. The size of the total binding capacity may vary with the feed conditions loaded onto the membrane.

Table 6 displays the binding capacity results for the cellulose acetate filters for the pre-TFF filtration condition.

TABLE 6
Binding capacity results for pre-TFF samples

| | | | | | | Binding |
|---|---|---|---|---|---|---|
| | | | | | HMW at | capacity |
| | | Protein | | HMW at | 70% RRT | HMW at |
| | HMW at | conc | Volume | 70% RRT | removed | 70% RRT |
| Sample ID | 70% RRT | (mg/mL) | (mL) | (mg) | (mg) | (mg/cm²) |
| Pre-TFF Material | 17.46 | 1.42 | 3.36 | 0.834 | 0.1969 | 0.044 |
| Run 1 (Lot #1) | | | | | | |
| Pool Sample 1 | | | | | | |
| (Load) | | | | | | |
| Pre-TFF Material | 15.20 | 0.77 | 5.44 | 0.637 | | |
| Run 1 (Lot #1) | | | | | | |
| 0.2 μm CA | | | | | | |
| Filtered Sample 1 | | | | | | |
| Pre-TFF Material | 17.78 | 1.42 | 7.06 | 1.784 | 0.3041 | 0.068 |
| Run 1 (Lot #1) | | | | | | |
| Pool Sample 2 | | | | | | |
| (Load) | | | | | | |
| Pre-TFF Material | 15.86 | 1.00 | 9.32 | 14.80 | | |
| Run 1 (Lot #1) | | | | | | |
| 0.2 μm CA | | | | | | |
| Filtered Sample 2 | | | | | | |
| Pre-TFF Material | 18.41 | 1.42 | 8.40 | 2.197 | 0.6039 | 0.134 |
| Run 1 (Lot #1) | | | | | | |
| Pool Sample 3 | | | | | | |
| (Load) | | | | | | |
| Pre-TFF Material | 15.17 | 1.06 | 9.91 | 1.594 | | |
| Run 1 (Lot #1) | | | | | | |
| 0.2 μm CA | | | | | | |
| Filtered Sample 3 | | | | | | |
| Pre-TFF Material | 7.81 | 1.34 | 3.36 | 0.352 | 0.1314 | 0.029 |
| Run 2 (Lot #2) | | | | | | |
| Pool Sample 1 | | | | | | |
| (Load) | | | | | | |
| Pre-TFF Material | 5.42 | 0.73 | 5.61 | 0.221 | | |
| Run 2 (Lot #2) | | | | | | |
| 0.2 μm CA | | | | | | |
| Filtered Sample 1 | | | | | | |
| Pre-TFF Material | 8.38 | 1.34 | 7.06 | 0.795 | 0.1399 | 0.031 |
| Run 2 (Lot #2) | | | | | | |
| Pool Sample 2 | | | | | | |
| (Load) | | | | | | |
| Pre-TFF Material | 7.31 | 0.98 | 9.11 | 0.655 | | |
| Run 2 (Lot #2) | | | | | | |
| 0.2 μm CA | | | | | | |
| Filtered Sample 2 | | | | | | |
| Pre-TFF Material | 8.52 | 1.34 | 8.40 | 0.961 | 0.2547 | 0.057 |
| Run 2 (Lot #2) | | | | | | |
| Pool Sample 3 | | | | | | |
| (Load) | | | | | | |
| Pre-TFF Material | 6.88 | 0.99 | 10.33 | 0.706 | | |
| Run 2 (Lot #2) | | | | | | |
| 0.2 μm CA | | | | | | |
| Filtered Sample 3 | | | | | | |

The loading capacities studied for the HMW at 70% RRT were between 0.078 mg and 0.488 mg HMW at 70% RRT/cm². The data in Table 6 show some variability between the different feed materials studied. The highest binding capacity was observed for the Lot #1 sample, with a binding capacity of 0.134 mg HMW 70% RRT/cm² when approximately 0.488 mg of HMW at 70% RRT/cm² was loaded. An increment of the binding capacity was observed for both the Lot #1 and Lot #2 samples loaded into the cellulose acetate filters, with an increase of the HMW at 70% RRT loaded. The binding capacity data shows a trend for the total HMW at 70% RRT bound to the cellulose acetate membrane, which increased with an incremental increase of the HMW at 70% RRT load. The loading conditions evaluated provide an indication that the membrane saturation capacity was not reached.

The average mg of HMW at 70% RRT bound to the cellulose acetate membrane was calculated to estimate the binding capacity for HMW aggregate under pre-TFF conditions. The average binding capacity from the experimental data for the pre-TFF material was 0.0605 mg HMW at 70% RRT/cm². This average binding capacity was used to estimate the filter requirements for GMP batches in pre-TFF buffer conditions. FIG. 1 is a graph illustrating the estimated binding capacities for various available cellulose acetate filter sizes based on the calculated average binding capacity, assuming linearity. It is noted that some operational parameters (e.g., backpressure, liquid flow path, flow rate, feed variability) could affect the membrane binding capacity on scale-up from laboratory scale to process scale.

### Post-TFF Filtration Step

### % HMW and Dimer Reduction

The SEC-HPLC and A280 characterisation of the flowthrough material for post-TFF (i.e., formulation buffer; 0.9% NaCl solution) samples is displayed in Table 7. The percent reduction for HMW at 70% RRT and dimer were normalised as a percentage reduction based on the SEC-HPLC HMW aggregate and dimer data. The loading capacity is provided as mg of HMW at 70% RRT/cm².

TABLE 7
HMW and dimer reduction results for post-TFF samples

| | Loading | | | | | Reduction | |
|---|---|---|---|---|---|---|---|
| | capacity | | | | | % | |
| | (mg HMW | % HMW | | | | HMW | |
| | at 70% | at 70% | | Dimer | Monomer | at 70% | % |
| Sample ID | RRT/cm²) | RRT (%) | LMW (%) | (%) | (%) | RRT^{a} | dimer |
| Lot #1 Control | 0.224 | 20.16 | 0.26 | 2.46 | 76.91 | 23.4% | −4.9% |
| Sample 1 | | | | | | | |
| Lot #1 0.2 μm | | 15.45 | 0.28 | 2.58 | 81.44 | | |
| Filtered | | | | | | | |
| Sample 1 | | | | | | | |
| Lot #1 Control | 0.458 | 20.60 | 0.27 | 2.49 | 76.44 | 12.0% | −3.2% |
| Sample 2 | | | | | | | |
| Lot #1 0.2 μm | | 18.13 | 0.28 | 2.57 | 78.79 | | |
| Filtered | | | | | | | |
| Sample 2 | | | | | | | |
| Lot #1 Control | 0.551 | 19.80 | 0.32 | 2.58 | 77.10 | 7.4% | 0.0% |
| Sample 3 | | | | | | | |
| Lot #1 0.2 μm | | 18.33 | 0.28 | 2.58 | 78.58 | | |
| Filtered | | | | | | | |
| Sample 3 | | | | | | | |
| Lot #4 Control | 0.113 | 10.73 | 0.51 | 3.02 | 85.47 | 69.8% | −7.9% |
| Sample 1 | | | | | | | |
| Lot #4 0.2 μm | | 3.24 | 0.63 | 3.26 | 92.56 | | |
| Filtered | | | | | | | |
| Sample 1 | | | | | | | |
| Lot #4 Control | 0.233 | 11.07 | 0.53 | 3.03 | 85.10 | 27.8% | −4.0% |
| Sample 2 | | | | | | | |
| Lot #4 0.2 μm | | 7.99 | 0.58 | 3.15 | 88.00 | | |
| Filtered | | | | | | | |
| Sample 2 | | | | | | | |
| Lot #4 Control | 0.296 | 11.26 | 0.54 | 3.03 | 84.89 | 37.4% | −5.3% |
| Sample 3 | | | | | | | |
| Lot #4 0.2 μm | | 7.05 | 0.59 | 3.19 | 88.87 | | |
| Filtered | | | | | | | |
| Sample 3 | | | | | | | |
| Lot #3 / Lot #4 | 0.204 | 9.74 | 0.59 | 3.26 | 86.21 | 35.2 | −4.3% |
| 1:1 Mixture | | | | | | | |
| Control | | | | | | | |
| Lot #3 / Lot #4 | | 6.31 | 0.66 | 3.40 | 89.43 | | |
| 1:1 Mixture | | | | | | | |
| 0.2 μm | | | | | | | |
| Filtered | | | | | | | |
| Sample | | | | | | | |

${}^{a} \% H M W at 70 \% RRT reduction = 100 - ( \frac{\% H M W at 70 \% RRT post ‐ filtration}{\% H M W at 70 \% RRT pre ‐ filtration} \times 100 )$

A difference in the reduction of the HMW at 70% RRT between samples from different batches was observed, which may be due to the differences in the feed composition (i.e., sample composition, sample concentration and HMW aggregate content). The reduction of HMW at 70% RRT for Lot #1 samples ranged from 7.4% to 23.4% for loading rates between 0.224 and 0.551 mg of HMW at 70% RRT/cm². For the Lot #4 samples, a higher reduction in HMW at 70% RRT was observed, ranging from 27.8% to 69.8% for loading rates between 0.113 and 0.296 mg of HMW at 70% RRT/cm². For the Lot #3/Lot #4 1:1 mixture sample, a reduction of 35% was observed when 0.204 mg of HMW at 70% RRT/cm² was loaded onto the membrane. A reduction of 23%-35% of HMW at 70% RRT was observed when similar quantities were loaded. The highest reduction of HMW at 70% RRT was observed for the experiment performed with a loading rate of 0.113 mg HMW at 70% RRT/cm² for the Lot #4 sample, resulting in a 70% reduction of HMW at 70% RRT.

No significant difference in dimer content was observed across the conditions studied. The differences in dimer observed between the pre- and post-filtration samples may be due to the slight enrichment of this species due to the removal of the HMW aggregate by the cellulose acetate filter.

### Recovery

Table 8 outlines the product recovery and the monomeric recovery obtained for each post-TFF condition evaluated. The monomeric recovery was calculated based on the mg of the monomer obtained pre-filtration and post-filtration using the purity values from the SEC-HPLC data.

TABLE 8
Recovery results for post-TFF samples

| | Loading | | | | | | |
|---|---|---|---|---|---|---|---|
| | Capacity | | Protein | | | | |
| | (g GmAb- | Volume | conc | Qty | | Recovery (%) | |
| Sample ID | DFO/m²) | (mL) | (mg/mL) | (mg) | Monomer | Overall | Monomer |
| Lot #1 Pool | 11.2 | 2.40 | 2.088 | 5.01 | 76.91 | 89.19% | 94.45% |
| Sample 1 | | | | | | | |
| (Load) | | | | | | | |
| Lot #1 0.2 μm | | 4.70 | 0.951 | 4.47 | 81.44 | | |
| Filtered | | | | | | | |
| Sample 1 | | | | | | | |
| Lot #1 Pool | 22.4 | 4.80 | 2.088 | 10.02 | 76.44 | 92.67% | 95.52% |
| Sample 2 | | | | | | | |
| (Load) | | | | | | | |
| Lot #1 0.2 μm | | 7.01 | 1.325 | 9.29 | 78.79 | | |
| Filtered | | | | | | | |
| Sample 2 | | | | | | | |
| Lot #1 Pool | 28 | 6.00 | 2.088 | 12.53 | 77.10 | 93.78% | 95.58% |
| Sample 3 | | | | | | | |
| (Load) | | | | | | | |
| Lot #1 0.2 μm | | 8.28 | 1.419 | 11.75 | 78.58 | | |
| Filtered | | | | | | | |
| Sample 3 | | | | | | | |
| Lot #4 Pool | 11.2 | 2.40 | 1.971 | 4.73 | 85.47 | 87.81% | 95.09% |
| Sample 1 | | | | | | | |
| (Load) | | | | | | | |
| Lot #4 0.2 μm | | 4.51 | 0.921 | 4.15 | 92.56 | | |
| Filtered | | | | | | | |
| Sample 1 | | | | | | | |
| Lot #4 Pool | 22.4 | 4.80 | 1.971 | 9.46 | 85.10 | 91.05% | 94.15% |
| Sample 2 | | | | | | | |
| (Load) | | | | | | | |
| Lot #4 0.2 μm | | 6.82 | 1.263 | 8.61 | 88.00 | | |
| Filtered | | | | | | | |
| Sample 2 | | | | | | | |
| Lot #4 Pool | 28 | 6.00 | 1.971 | 11.83 | 84.89 | 94.25% | 98.67% |
| Sample 2 | | | | | | | |
| (Load) | | | | | | | |
| Lot #4 0.2 μm | | 8.10 | 1.376 | 11.15 | 88.87 | | |
| Filtered | | | | | | | |
| Sample 2 | | | | | | | |
| Lot #3 / Lot #4 | 22.4 | 4.80 | 1.962 | 9.42 | 86.21 | 92.52% | 95.98% |
| 1:1 Mixture | | | | | | | |
| Pool (Load) | | | | | | | |
| Lot #3 / Lot #4 | | 6.91 | 1.261 | 8.71 | 89.43 | | |
| 1:1 Mixture | | | | | | | |
| 0.2 μm | | | | | | | |
| Filtered | | | | | | | |
| Sample | | | | | | | |

No significant differences in terms of monomeric recoveries were observed between each loading condition evaluated for the cellulose acetate filters. The overall percent recoveries ranged from 87.81% to 94.25%; the overall recovery may be expected to be lower due to the removal of HMW aggregate in HMW aggregate containing samples. Monomeric recoveries ranged from 94.15% to 98.67%. High monomeric recoveries were observed for all the load conditions and materials evaluated. The recovery losses may be due to the system hold up volume of the bench scale for the post-TFF samples passed through the cellulose acetate filter (the volume retained in the system and filter without air purge). No significant losses of monomer were observed indicating no significant interaction between the monomer and the cellulose acetate membrane. Based on these results, it may be beneficial to optimise the flush accordingly for the cellulose acetate filtration step for process scale.

### Binding Capacity Results

The total HMW aggregate binding capacity for the cellulose acetate membrane was estimated in batch mode and is considered the maximum amount of HMW at 70% RRT able to bind to the membrane medium under the feed and buffer conditions evaluated.

Table 9 displays the binding capacity results for the cellulose acetate filters for the post-TFF filtration condition.

TABLE 9
Binding capacity results for post-TFF samples

| | | | | | | Binding |
|---|---|---|---|---|---|---|
| | | | | | HMW at | capacity |
| | | Protein | | HMW at | 70% RRT | HMW at |
| | HMW at | conc | Volume | 70% RRT | removed | 70% RRT |
| Sample ID | 70% RRT | (mg/mL) | (mL) | (mg) | (mg) | (mg/cm²) |
| Lot #1 Control | 20.16 | 2.09 | 2.40 | 1.010 | 0.3183 | 0.071 |
| Sample 1 (Load) | | | | | | |
| Lot #1 0.2 μm | 15.48 | 0.95 | 4.70 | 0.692 | | |
| Filtered Sample 1 | | | | | | |
| Lot #1 Control | 20.60 | 2.09 | 4.80 | 2.067 | 0.3826 | 0.085 |
| Sample 2 (Load) | | | | | | |
| Lot #1 0.2 μm | 18.13 | 1.33 | 7.01 | 1.684 | | |
| Filtered Sample 2 | | | | | | |
| Lot #1 Control | 19.80 | 2.09 | 6.00 | 2.483 | 0.3293 | 0.073 |
| Sample 3 (Load) | | | | | | |
| Lot #1 0.2 μm | 18.33 | 1.42 | 8.28 | 2.154 | | |
| Filtered Sample 3 | | | | | | |
| Lot #4 Control | 10.73 | 1.97 | 2.40 | 0.507 | 0.3727 | 0.083 |
| Sample 1 (Load) | | | | | | |
| Lot #4 0.2 μm | 3.24 | 0.92 | 4.51 | 0.135 | | |
| Filtered Sample 1 | | | | | | |
| Lot #4 Control | 11.07 | 1.97 | 4.80 | 1.047 | 0.3585 | 0.080 |
| Sample 2 (Load) | | | | | | |
| Lot #4 0.2 μm | 7.99 | 1.26 | 6.82 | 0.688 | | |
| Filtered Sample 2 | | | | | | |
| Lot #4 Control | 11.26 | 1.97 | 6.00 | 1.331 | 0.5452 | 0.121 |
| Sample 3 (Load) | | | | | | |
| Lot #4 0.2 μm | 7.05 | 1.38 | 8.10 | 0.786 | | |
| Filtered Sample 3 | | | | | | |
| Lot #3 / Lot #4 | 9.74 | 1.96 | 4.80 | 0.916 | 0.3665 | 0.081 |
| 1:1 Mixture | | | | | | |
| Control (Load) | | | | | | |
| Lot #3 / Lot #4 | 6.31 | 1.26 | 6.91 | 0.550 | | |
| 1:1 Mixture 0.2 | | | | | | |
| μm Filtered | | | | | | |
| Sample | | | | | | |

The loading capacities observed for the HMW aggregate were between 0.113 mg and 0.551 mg HMW at 70% RRT/cm². The data in Table 9 for the Lot #1 material may indicate filter saturation around 0.085 mg of HMW at 70% RRT/cm² under the conditions studied for this sample. However, for the Lot #4 sample, a higher binding capacity of 0.121 mg of HMW at 70% RRT/cm² was observed. This may provide an indication that the saturation point of the membrane may depend on the feed material. The loading conditions evaluated for the Lot #4 sample provide an indication that the membrane saturation capacity was not reached.

The average mg of HMW at 70% RRT bound to the membrane was calculated to estimate the binding capacity of the filter for the post-TFF condition, with the view of estimating a suitable filter size to use at process scale based on previous GMP experience. The average binding capacity from the experimental data for the post-TFF material was 0.0849 mg HMW at 70% RRT/cm². FIG. 2 is a graph illustrating the estimated binding capacities for various available cellulose acetate filter sizes based on based on the calculated average binding capacity, assuming linearity. It is noted that some operating parameters (e.g., backpressure, liquid flow path, flow rate, feed variability) could affect the membrane binding capacity on scale-up from laboratory scale to process scale.

### Characterisation

The samples were characterised by SEC-HPLC and A280 using Nanodrop equipment. An aliquot of each sample was placed in the −80° C. freezer for 24 hours and then transferred to a −20° C. freezer until further characterisation if needed.

### A280

The concentration of the samples was determined by absorbance at 280 nm. System suitability measurements were performed on the Nanodrop using a bovine serum albumin (BSA) standard. The system suitability measurements passed all acceptance criteria of BSA concentration within the range of 0.95-1.05 mg/mL at the beginning and end of each run, confirming that the NanoDrop instrument was performing suitably. The Nanodrop was blanked using PBS buffer, pH 7.1. The concentration of the GmAb-DFO sample was calculated using the extinction coefficient of 1.35 (mg/mL)⁻¹ cm⁻¹ and the following equation (1):

$$GmAb Concentration , \frac{mg}{ml} = \frac{A 280 Value}{( 1.35 \frac{{mg}^{- 1}}{mL} {cm}^{- 1} ) * ( 1 cm )}$$

Table 10 outlines the results of the protein concentration by A280.

TABLE 10
Protein concentration by A280

| | | Protein conc | Average protein |
|---|---|---|---|
| Sample ID | A280 | (mg/mL) | conc (mg/mL) |
| Lot #1 Pool (Load) | 2.824 | 2.092 | 2.09 |
| | 2.827 | 2.094 | |
| | 2.805 | 2.078 | |
| Lot #1 Control 1 | 1.368 | 1.014 | 1.02 |
| | 1.39 | 1.029 | |
| | 1.383 | 1.025 | |
| Lot #1 0.2 μm Filtered Sample 1 | 1.288 | 0.954 | 0.95 |
| | 1.286 | 0.953 | |
| | 1.276 | 0.945 | |
| Lot #1 Control 2 | 1.862 | 1.379 | 1.38 |
| | 1.863 | 1.38 | |
| | 1.857 | 1.376 | |
| Lot #1 0.2 μm Filtered Sample 2 | 1.797 | 1.331 | 1.32 |
| | 1.789 | 1.325 | |
| | 1.779 | 1.318 | |
| Lot #1 Control 3 | 1.986 | 1.471 | 1.47 |
| | 1.998 | 1.48 | |
| | 1.985 | 1.47 | |
| Lot #1 0.2 μm Filtered Sample 3 | 1.92 | 1.422 | 1.42 |
| | 1.917 | 1.42 | |
| | 1.91 | 1.415 | |
| Lot #4 Pool (Load) | 2.635 | 1.952 | 1.97 |
| | 2.673 | 1.98 | |
| | 2.675 | 1.981 | |
| Lot #4 Control 1 | 1.354 | 1.003 | 1.00 |
| | 1.353 | 1.002 | |
| | 1.35 | 1.0000 | |
| Lot #4 0.2 μm Filtered Sample 1 | 1.235 | 0.915 | 0.92 |
| | 1.243 | 0.921 | |
| | 1.251 | 0.926 | |
| Lot #4 Control 2 | 1.836 | 1.36 | 1.36 |
| | 1.836 | 1.36 | |
| | 1.829 | 1.355 | |
| Lot #4 0.2 μm Filtered Sample 2 | 1.707 | 1.265 | 1.26 |
| | 1.711 | 1.267 | |
| | 1.695 | 1.256 | |
| Lot #4 Control 3 | 1.943 | 1.439 | 1.44 |
| | 1.944 | 1.44 | |
| | 1.943 | 1.439 | |
| Lot #4 0.2 μm Filtered Sample 3 | 1.854 | 1.373 | 1.38 |
| | 1.86 | 1.378 | |
| | 1.857 | 1.376 | |
| Lot #3 / Lot #4 1:1 Mixture Pool | 2.649 | 1.962 | 1.96 |
| (Load) | 2.657 | 1.968 | |
| | 2.642 | 1.957 | |
| Lot #3 / Lot #4 1:1 Mixture | 1.838 | 1.361 | 1.36 |
| Control | 1.826 | 1.353 | |
| | 1.823 | 1.351 | |
| Lot #3 / Lot #4 1:1 Mixture 0.2 | 1.704 | 1.262 | 1.26 |
| μm Filtered Sample | 1.699 | 1.258 | |
| | 1.706 | 1.264 | |
| Pre-TFF Material Run 1 (Lot #1) | 1.812 | 1.342 | 1.34 |
| Pool (Load) | 1.814 | 1.343 | |
| | 1.816 | 1.345 | |
| Pre-TFF Material Run 1 (Lot #1) | 1.099 | 0.814 | 0.80 |
| Control 1 | 1.083 | 0.802 | |
| | 1.077 | 0.798 | |
| Pre-TFF Material Run 1 (Lot #1) | 1.045 | 0.774 | 0.77 |
| 0.2 μm CA Filtered Sample 1 | 1.035 | 0.767 | |
| | 1.04 | 0.77 | |
| Pre-TFF Material Run 1 (Lot #1) | 1.362 | 1.009 | 1.00 |
| Control 2 | 1.35 | 1 | |
| | 1.338 | 0.991 | |
| Pre-TFF Material Run 1 (Lot #1) | 1.355 | 1.004 | 1.01 |
| 0.2 μm CA Filtered Sample 2 | 1.368 | 1.013 | |
| | 1.354 | 1.003 | |
| Pre-TFF Material Run 1 (Lot #1) | 1.499 | 1.11 | 1.11 |
| Control 3 | 1.505 | 1.115 | |
| | 1.494 | 1.107 | |
| Pre-TFF Material Run 1 (Lot #1) | 1.434 | 1.062 | 1.06 |
| 0.2 μm CA Filtered Sample 3 | 1.432 | 1.06 | |
| | 1.429 | 1.058 | |
| Pre-TFF Material Run 2 (G-434- | 1.92 | 1.422 | 1.42 |
| 901-DSI) Pool | 1.91 | 1.415 | |
| | 1.925 | 1.426 | |
| Pre-TFF Material Run 2 (Lot #2) | 1.031 | 0.763 | 0.77 |
| Control 1 | 1.05 | 0.778 | |
| | 1.036 | 0.768 | |
| Pre-TFF Material Run 2 (Lot #2) | 0.981 | 0.727 | 0.73 |
| 0.2 μm CA Filtered Sample 1 | 0.984 | 0.729 | |
| | 0.979 | 0.725 | |
| Pre-TFF Material Run 2 (Lot #2) | 1.323 | 0.98 | 0.98 |
| Control 2 | 1.329 | 0.984 | |
| | 1.32 | 0.978 | |
| Pre-TFF Material Run 2 (Lot #2) | 1.327 | 0.983 | 0.98 |
| 0.2 μm CA Filtered Sample 2 | 1.324 | 0.981 | |
| | 1.329 | 0.984 | |
| Pre-TFF Material Run 2 (Lot #2) | 1.488 | 1.102 | 1.10 |
| Control 3 | 1.479 | 1.096 | |
| | 1.49 | 1.103 | |
| Pre-TFF Material Run 2 (Lot #2) | 1.344 | 0.996 | 0.99 |
| 0.2 μm CA Filtered Sample 3 | 1.34 | 0.993 | |
| | 1.34 | 0.992 | |

### SEC-HPLC

Samples were analysed using an Agilent 1200 series HPLC system equipped with a Yarra SEC-300 (3 μm, 290 Å, 7.8×300 mm) SE-HPLC column. Data were analysed by comparing the area of each protein species to the total area detected. No significant variation in the relative retention time for the monomer, HMW at 70% RRT, HMW and dimer was observed.

### Filter Evaluation in GMP Process

Filtration steps using cellulose acetate 0.2 μm filters (Sartobran MidiCaps 0.05 m², Sartorius) were added to a conjugation reaction for preparing GmAb-DFO at process scale both before and after the TFF step. In brief, a solution of staring material girentuximab (GmAb; GmAb product pool) was adjusted to pH 9.6 to provide the pH Adjusted GmAb Pool. A solution of TFP-N-sucDf-Fe was prepared in acetonitrile and added to the pH Adjusted GmAb Pool. The reaction was incubated at ambient temperature for 30±2 min to provide the Conjugated Product Pool. The Conjugated Product Pool was adjusted to pH 7.0, heated to 35° C., and further titrated to pH 4.4 to provide the Low pH Conjugated Product Pool. Iron was removed from GmAb-N-sucDf-Fe by the addition of disodium EDTA. The reaction was incubated at 35° C. to provide the Transchelated Product Pool. The Transchelated Product Pool was filtered using a 0.05 m² Sartobran cellulose acetate filter (0.2 μm) to provide the Filtered Transchelated Pool. The filter was flushed with 0.9% NaCl. The Filtered Transchelated Product Pool was then subjected to a UFDF process into 0.9% NaCl to provide the UFDF Conjugation Pool. The UFDF Conjugation Pool was filtered using a 0.05 m² Sartobran cellulose acetate filter (0.2 μm) to provide the Filtered UFDF Conjugation Pool. The filter was flushed with 0.9% NaCl to ensure complete recovery of the product to yield the Filtered UFDF Conjugation Pool. To provide the final BDS material, the Filtered UFDF Conjugation Pool was passed through a 0.01 m² Millipak 20 (0.2 μm) filter, with a PVDF membrane, as a final bioburden reduction step. Four batches were evaluated: batch #1; batch #2; batch #3; batch #4.

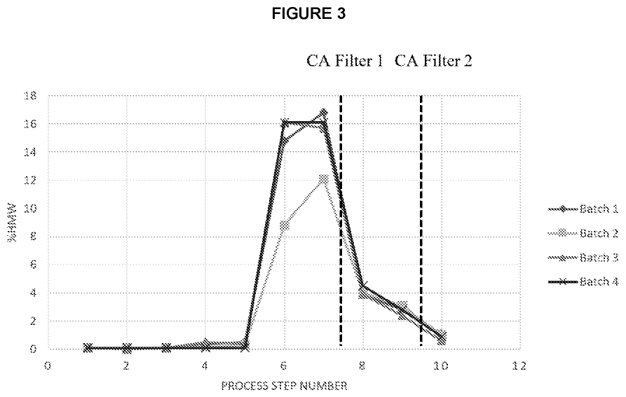

In-process SEC-HPLC results from these batches are displayed in FIG. 3 and summarised in Table 11. These data show that the pre-TFF cellulose acetate filter was consistently able to reduce the HMW aggregate content from ˜15% to ˜4% across all four batches, whereas the post-TFF cellulose acetate filter provided an HMW aggregate “polish” reducing the HMW content from ˜3% to ˜1%. In all four batches analysed, the cellulose acetate filtration strategy was able to effectively control the HMW aggregate levels to a satisfactory within-specification level with a high (>85%-95%) monomeric recovery. This demonstrates that the cellulose acetate filter is useful for removing HMW aggregate under different conditions during a process scale conjugation reaction.

TABLE 11
In-process SEC-HPLC data for process scale GmAb-DFO
batches

| | | GMP batch no | | | |
|---|---|---|---|---|---|
| | | Batch | Batch | Batch | Batch |
| In-process sample | % Purity | #1 | #2 | #3 | #4 |
| GmAb Product Pool | Monomer | 98.4 | 98.7 | 98.3 | 98.0 |
| | HMW 70% | 0.1 | 0.1 | 0.1 | 0.1 |
| | RRT | | | | |
| | Dimer | 0.9 | 0.8 | 0.9 | 1.1 |
| pH Adjusted GmAb | Monomer | 98.4 | 98.6 | 98.3 | 98.0 |
| Pool | HMW 70% | 0.1 | 0.1 | 0.0 | 0.1 |
| | RRT | | | | |
| | Dimer | 0.9 | 0.8 | 0.9 | 1.1 |
| Conjugated Product | Monomer | 98.4 | 98.7 | 98.3 | 98.0 |
| Pool | HMW 70% | 0.1 | 0.1 | 0.1 | 0.1 |
| | RRT | | | | |
| | Dimer | 0.8 | 0.8 | 0.9 | 1.1 |
| pH Adjusted | Monomer | 98.0 | 98.4 | 98.0 | 97.9 |
| Conjugated | HMW 70% | 0.4 | 0.2 | 0.5 | 0.1 |
| Product | RRT | | | | |
| Pool | Dimer | 0.9 | 0.9 | 0.9 | 1.2 |
| Temperature | Monomer | 97.9 | 98.3 | 98.0 | 97.9 |
| Adjusted | | | | | |
| Conjugated | HMW 70% | 0.5 | 0.3 | 0.4 | 0.1 |
| Product | RRT | | | | |
| Pool | Dimer | 0.9 | 0.9 | 0.9 | 1.2 |
| Low pH Conjugated | Monomer | 83.8 | 89.8 | 82.4 | 82.4 |
| Product Pool | HMW 70% | 14.8 | 8.8 | 16.1 | 16.1 |
| | RRT | | | | |
| | Dimer | 0.9 | 0.8 | 0.8 | 0.8 |
| Transchelated | Monomer | 81.8 | 86.6 | 82.9 | 82.5 |
| Product Pool | HMW 70% | 16.8 | 12.1 | 15.7 | 16.1 |
| | RRT | | | | |
| | Dimer | 0.8 | 0.8 | 0.8 | 0.7 |
| Filtered | Monomer | 94.4 | 94.6 | 94.5 | 94.0 |
| Transchelated Pool | HMW 70% | 3.9 | 4.0 | 3.9 | 4.5 |
| | RRT | | | | |
| | Dimer | 1.0 | 0.9 | 0.9 | 0.9 |
| UFDF Conjugation | Monomer | 95.6 | 95.7 | 96.3 | 95.8 |
| Pool | HMW 70% | 2.8 | 3.1 | 2.4 | 2.8 |
| | RRT | | | | |
| | Dimer | 1.1 | 0.9 | 1.0 | 0.9 |
| Filtered UFDF | Monomer | 97.2 | 97.8 | 98.0 | 97.8 |
| Conjugation Pool | HMW 70% | 1.1 | 1.0 | 0.6 | 0.9 |
| | RRT | | | | |
| | Dimer | 1.1 | 1.0 | 1.0 | 0.9 |

### Example 3. Removal of Aggregates of HuJ591-DOAT-177Lu

A sample of aggregated HuJ591-DOTA was created by adjusting the pH of the HuJ591-DOTA material to pH 4.0 and incubating at 35° C. for 60 min.

60 μL of aggregated HuJ591-DOTA was added to 50 μL of 177Lu/HCl and incubated at 35° C. for 35 min to generate aggregated 177Lu-DOTA-HuJ591.

110 μL of the aggregated 177Lu-DOTA-HuJ591 was passed through a cellulose acetate 0.22 μm filter.

The material was analysed by A280 (nanodrop) and SEC-HPLC pre- and post-filtration (see Table 12).

TABLE 12
Summary of analysis of 177Lu-DOTA-HuJ591 before and after cellulose acetate filtration

| | | | Total | | | | | | |
|---|---|---|---|---|---|---|---|---|---|
| | Volume | | Mass | | | | | % recovery | % reduction |
| Sample | (mL) | mg/mL | (mg) | % Mono | % Dimer | % HMW | % recovery | (mono) | (HMW) |
| 177Lu- | 0.11 | 0.98 | 0.1078 | 67.05 | 10.54 | 22.41 | 90.9 | 99.7 | 30.4 |
| DOTA- | | | | | | | | | |
| HuJ591 | | | | | | | | | |
| (pre | | | | | | | | | |
| filter) | | | | | | | | | |
| 177Lu- | 0.10 | 0.98 | 0.098 | 73.60 | 9.25 | 17.15 | | | |
| DOTA- | | | | | | | | | |
| HuJ591 | | | | | | | | | |
| (post | | | | | | | | | |
| filter) | | | | | | | | | |

1. A method of removing an aggregate of a protein from a composition comprising the protein, the aggregate of the protein and a liquid carrier, the method comprising:
subjecting the composition to one or more filtering steps comprising passing the composition through a cellulose acetate membrane to selectively adsorb at least some of the aggregate onto the membrane while substantially allowing the protein to pass through the membrane.

2. The method of claim 1, wherein the protein comprises a conjugated chemical moiety.

3. The method of claim 2, wherein the conjugated chemical moiety comprises a chelating ligand.

4. The method of claim 3, wherein the chelating ligand is selected from desferrioxamine (DFO) and 1,4,7,10-tetraazacyclododecane-N,N′,N″,N′-tetraacetic acid (DOTA).

5. The method of any one of claims 1-4, wherein the protein comprises a protein targeting agent.

6. The method of any one of claims 1-5, wherein the protein comprises an antibody.

7. The method of any one of claims 1-4, wherein the protein comprises girentuximab or HuJ591.

8. The method of any one of claims 1-7, wherein the cellulose acetate membrane has a size of about 0.015 m² to about 0.6 m².

9. The method of any one of claims 1-8, wherein the cellulose acetate membrane comprises pores having an average diameter of about 0.2 μm to about 0.8 μm.

10. The method of any one of claims 1-9, wherein the liquid in each filtering step independently has a pH of about 4.0 to about 8.0.

11. The method of any one of claims 1-10, wherein the liquid carrier in each filtering step is independently an aqueous solution.

12. The method of any one of claims 1-11, wherein the liquid carrier in each filtering step is independently a buffer solution.

13. The method of any one of claims 1-12, wherein each of the filtering steps independently reduces the aggregate content in the composition by about 0.02 mg/cm² to about 0.15 mg/cm², relative to the size of the cellulose acetate membrane.

14. The method of any one of claims 1-13, wherein each of the filtering steps independently reduces the aggregate content in the composition to not more than about 10%, relative to the total protein content of protein species in the composition.

15. The method of any one of claims 1-14, wherein the method is not for the removal of particulate matter or bioburden.

16. The method of any one of claims 1-15, wherein the composition is obtained from a process for preparing a protein conjugate in which an undesired aggregate is formed.

17. A protein obtainable or obtained by the method according to any one of claims 1-16.

18. A cellulose acetate membrane for removing an aggregate of a protein from a composition comprising the protein, the aggregate of the protein and a liquid carrier; and/or selectively adsorbing an aggregate of a protein from a composition comprising the protein, the aggregate of the protein and a liquid carrier.
